(19) United States
(12) Patent Application Publication
(10) Pub. No.: US 2011/0086825 A1
(43) Pub. Date: Apr. 14, 2011

(54) Therapeutic vaginal emollient

(76) Inventor: Sylvia S. Chatroux, Ashland, OR (US)

(21) Appl. No.: 12/587,755

(22) Filed: Oct. 13, 2009

(51) Int. Cl. A61K 31/56

(52) U.S. Cl. 514/170

(57) ABSTRACT

A topical composition for relief from certain menopausal, peri- and post-menopausal symptoms is disclosed, consisting of a low dosage of progesterone, testosterone and estriol in a pharmaceutically acceptable topical carrier in a weight ratio of from about 1:0.1:0.01 to about 1:0.1:0.02, respectively. In a preferred single dose, the three active ingredients are present as follows: (a) from about 20 to about 35 mg progesterone; (b) from about 2 to about 3.5 mg testosterone; and (c) from about 0.2 to about 0.7 mg estriol. A preferred carrier is cocoa butter, and a preferred single dose form is a vaginal suppository.

### BACKGROUND OF THE INVENTION

As a woman approaches the age of 50, give or take a few years, she begins to stop menstruating, entering a period of time known as menopause. The time leading up to and just past menopause, known as the peri-menopausal period, can last many years. During this peri-menopausal period, due to the aging of the ovaries, the woman has progressively reduced levels of the hormones progesterone, testosterone and estrogen, which can contribute to any or all of the following symptoms/medical problems: bone loss, heart disease, weight gain, mood dysfunction, memory dysfunction, vaginal dryness, vaginal atrophy, thinning of the vaginal wall, frequent urinary tract infections and reduction or loss of libido. Such symptoms continue to a greater or lesser degree in post-menopausal women: Some of these symptoms can greatly affect sexual relations.

Hormone Replacement Therapy (HRT), or systemic dosing with pharmaceutical compositions containing one or more of those three hormones, can provide relief from such symptoms, but the possibility of adverse side effects exists, as with any systemically absorbed medication. In the case of HRT, estrogens have been well-studied and found to have a direct link to development of new breast cancers. Systemic estrogen therapy is contraindicated in any woman who has had breast cancer and is in recovery. The risk increases more in women who also take progesterone systemically. Use of systemic testosterone is controversial because it has been associated with an increase in the development of heart disease, as well as breast and ovarian cancer. Systemic hormone therapy in general can interfere with liver function.

A topical formulation would not result in systemic uptake of the hormones, thereby avoiding altogether the negative side effects mentioned above. Additionally, it may be dosed directly to the site of need. Currently there are topical creams available for treatment of vaginal dryness and the prevention of recurrent urinary tract infections associated with menopause. But such creams do not provide a non-systemic solution for the lack of libido that is associated with the menopausal, peri- and post-menopausal years.

There is therefore a need in the art for a form of low dose topical hormone therapy that alleviates the menopausal, peri- and post-menopausal symptoms of low libido and vaginal atrophy. This need is met by the present invention, which is summarized and described in detail below.

### BRIEF SUMMARY OF THE INVENTION

According to the invention, there is provided a low-dose composition of progesterone, testosterone and estriol in a pharmaceutically acceptable topical carrier in the weight ratios of progesterone:testosterone:estriol of from about 1:0.1:0.01 to about 1:0.1:0.02. In a preferred embodiment single dose, the composition comprises those three hormones in the following weights: (a) from about 20 to about 35 mg progesterone; (b) from about 2 to about 3.5 mg testosterone; and (c) from about 0.2 to about 0.7 mg estriol. A preferred single dose composition has the three components present in the topical carrier in the following weights: (a) about 20 mg progesterone; (b) about 3 mg testosterone; and (c) about 0.3 mg estriol.

### DETAILED DESCRIPTION OF PREFERRED EMBODIMENTS

As used herein, the term “about” means+10% of the stated number or parameter. Thus, for example, “about 20 mg” means 18 to 22 mg.

When used topically, the basic composition summarized above has been found to be remarkably effective in alleviating the menopausal, peri- and post-menopausal symptoms described above, in particular, vaginal dryness and decline in libido, yet has none of the side effects typically associated with the systemic administration of female hormones. And because of the very low dosage of the three hormones directly to the vaginal vault, systemic uptake is negligible.

The phrase “pharmaceutically acceptable” refers to molecular entities and compositions that are physiologically tolerable and do not typically produce an allergic or similar untoward reaction, such as rash, lesions and the like, when administered. Preferably, as used herein, the term “pharmaceutically acceptable” means approved by a regulatory agency of the Federal or a state government or listed in the U.S. Pharmacopoeia or other generally recognized pharmacopoeia for drug excipients used in humans.

Preferred pharmaceutically acceptable topical carriers include those that are stable at room temperature, in general those having a melting point of from about 25 to about 35° C. or are liquid at about 20° C. Examples include, without limitation, cocoa butter, polyethylene glycols (PEG), sorbitan fatty acid esters, polyoxyethylene fatty acid esters, polyoxyethylene stearates, aloe vera gel, aloe vera cream, petroleum jelly, glycerin, lanolin, vegetable oils and vitamin E, with cocoa butter being most preferred. As to PEG, PEG 1450 and PEG 1000 may be used in a 1:1 ratio to make a suppository that melts at or below body temperature.

While virtually any composition that may be applied topically is acceptable, a particularly preferred form of the composition is a vaginal suppository that releases said component in the vaginal cavity. Many methods may be used for vaginal administration of the formulation of the invention. These include vaginal administration of creams, suppositories, foams, gels (including but not limited to aqueous solutions and suspensions) ointments, tablets, ovules, pessaries and rings. A particularly preferred form of the composition is as a vaginal suppository of suitable size and shape for self-administration, and which is made as noted in the following Example.

### Example

A composition of the invention in the form of a vaginal suppository was made by combining 20 mg progesterone, 3 mg testosterone and 0.3 mg estriol with 200 mg of melted pharmaceutical grade cocoa butter to form a mixture, followed by pouring the molten mixture into a disc-shaped resin mold and allowing the mixture to cool for about an hour to room temperature (about 23° C.). The mold was opened and the so-formed suppository was retrieved and wrapped in foil packaging and stored under refrigeration to prevent the carrier from melting in the event ambient temperature exceeded 30° C.

Suppositories made in the foregoing fashion were prescribed by a physician to 20 menopausal, peri- and post-menopausal woman ranging in age from 45 to 75 years old to address a number of women's health issues, including vaginal dryness and thinning of the vaginal wall due to atrophy, recurring urinary tract infections and loss of libido. The suppositories were self-administered over a period of 12 weeks at a dosage rate of 2-3 per week. Based upon the reports given in follow-up visits with the prescribing physician, all of the women reported relief from the discomfort caused by the above mentioned symptoms, as well as partial restoration of libido, resulting in an increase in sexual relations.

The terms and expressions which have been employed in this specification are used as terms of description and not of limitation, and there is no intention in the use of such terms and expressions to exclude equivalents of the features shown and described or portions thereof, it being recognized that the scope of the invention is defined and limited only by the claims which follow.

1. A composition consisting essentially of the components progesterone, testosterone and estriol in a pharmaceutically acceptable topical carrier in the weight ratio of progesterone:testosterone:estriol of from 1:0.1:0.01 to 1:0.1:0.02.

2. The composition of claim 1 in a single dose wherein said components are present in the following amounts:
(a) from about 20 to about 35 mg progesterone;
(b) from about 2 to about 3.5 mg testosterone; and
(c) from about 0.2 to about 0.7 mg estriol.

3. The composition of claim 2 wherein said components are present in the following weights:
(a) about 20 mg progesterone;
(b) about 3 mg testosterone; and
(c) about 0.3 mg estriol.

4. The composition of claim 1 wherein said carrier is selected from the group consisting of cocoa butter, polyethylene glycols, sorbitan fatty acid esters, polyoxyethylene fatty acid esters, polyoxyethylene stearates, aloe vera gel, aloe vera cream, petroleum jelly, glycerin, lanolin, vegetable oils and vitamin E.

5. The composition of claim 4 wherein said carrier has a melting point of from about 25 to about 35° C.

6. The composition of claim 4 wherein said carrier is liquid at about 20° C.

7. The composition of claim 2 wherein said carrier is cocoa butter.

8. The composition of claim 7 wherein said cocoa butter is present in said composition from about 100 to about 300 mg.

9. The composition of claim 8 in the form of a vaginal suppository.

10. A method of administering a therapeutic dose of progesterone, testosterone and estriol to a human female comprising administering the composition of claim 1 into the vaginal vault.

11. A method of administering a therapeutic dose of progesterone, testosterone and estriol to a human female comprising administering the composition of claim 2 into the vaginal vault.

12. The method of claim 11 wherein the form of said composition is a vaginal suppository.

13. The method of claim 12 wherein said carrier of said composition is cocoa butter.

14. The method of claim 13 wherein said cocoa butter is present in an amount from about 100 to about 300 mg.
